SEQUENCE LISTING (17 sequences)

SEQ ID NO: 1 - protein, 178 aa, synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 178 aa, synthetic construct
MHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 3 - protein, 5 aa, synthetic construct
DTYIH

SEQ ID NO: 4 - protein, 5 aa, synthetic construct
RIYPT

SEQ ID NO: 5 - protein, 11 aa, synthetic construct
WGGDGFYAMDY

SEQ ID NO: 6 - protein, 11 aa, synthetic construct
RASQDVNTAVA

SEQ ID NO: 7 - protein, 7 aa, synthetic construct
SASFLYS

SEQ ID NO: 8 - protein, 9 aa, synthetic construct
QQHYTTPPT

SEQ ID NO: 9 - protein, 120 aa, synthetic construct [sequence omitted]

SEQ ID NO: 10 - protein, 107 aa, synthetic construct [sequence omitted]

SEQ ID NO: 11 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 12 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 567 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 15 - protein, 567 aa, synthetic construct
SPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRNGGGGSGGGGSGGGGSSPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRNGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 16 - protein, 451 aa, synthetic construct
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 214 aa, synthetic construct
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0022190 A1
(43) Pub. Date: Jan. 22, 2026

(54) HETERODIMERIC FUSION PROTEIN AND USE THEREOF

(71) Applicant: SHENGHE (CHINA) BIOPHARMACEUTICAL CO., LTD., Nanjing, Jiangsu (CN)

(72) Inventors: Chong ZHOU, Nanjing (CN); Liusong YIN, Nanjing (CN); Xiaoling JIANG, Nanjing (CN)

(73) Assignee: SHENGHE (CHINA) BIOPHARMACEUTICAL CO., LTD., Nanjing, Jiangsu (CN)

(21) Appl. No.: 18/881,400

(22) PCT Filed: Jul. 3, 2023
(86) PCT No.: PCT/CN2023/105535

§ 371 Date: Jan. 6, 2025

(30) Foreign Application Priority Data
Jul. 8, 2022 (CN) 202210801296.1

(51) Int. Cl. C07K 16/32; A61K 38/00; C07K 14/54

(52) U.S. Cl. CPC C07K 16/32; C07K 14/5428; A61K 38/00; C07K 2317/565; C07K 2319/00

(57) ABSTRACT

A heterodimeric fusion protein and use thereof. The heterodimeric fusion protein includes: a first heavy chain, wherein the first heavy chain includes an Fc region, an immunomodulator fused to the Fc region; a light chain and a second heavy chain, wherein the light chain and the second heavy chain are complexed to form a targeting portion exhibiting binding specificity to a tumor antigen or an immune checkpoint; and the light chain, the first heavy chain, and the second heavy chain are complexed to form a heterodimeric fusion protein, wherein the immunomodulator in the first heavy chain may contain a mutation. The heterodimeric fusion protein has high affinity for both tumor-associated targeting antigens and IL-10 receptors and has good anti-tumor activity.

### TECHNICAL FIELD

The present invention belongs to the field of biomedical technology, and relates to a heterodimeric fusion protein and use thereof.

### BACKGROUND

IL-10 is primarily secreted by activated T cells and antigen-presenting cells. During the process of antigen recognition, the expression of IL-10 receptor (IL-10R) in CD8+ T cells is upregulated. IL-10 mediates various activities through a specific cell surface receptor complex. The IL-10 receptor consists of two distinct chains, IL-10R1 and IL-10R2, both belonging to the class II cytokine receptor family (CRF2). In bacterial infections and tissue injury, IL-10 can reduce inflammatory responses, inhibit inflammation caused by T cells (Th17) and macrophages (IL-12/23), and decrease tumor-related inflammatory reactions. In the tumor microenvironment, IL-10 can efficiently activate the proliferation and toxicity of antigen-specific CD8+ T cells.

The mechanism of action of IL-10 in anti-tumor therapy is as follows: a. It can activate and expand the activity of CD8+ T cells within the tumor; b. IL-10 can increase the activity and expansion of antigen-specific T lymphocytes within the tumor; c. IL-10 has a memory function in tumor rejection. Animal experimental data indicate that after administering IL-10, the tumor disappears. When the mice are re-injected with tumor cells, the tumor cells do not grow in the mice. The main reason is that IL-10 can enhance the survival rate of antigen-specific CD8+ T cells, functioning as a tumor vaccine; d. IL-10 reactivates T cells by restoring oxidative phosphorylation metabolism in terminal exhausted T cells to achieve the purpose of killing tumor cells. Clinical trials have also shown that when combined with PDL1 antibodies, it increases the number of PDL1-specific CD8+ positive cells within the tumor, producing a lasting anti-tumor effect. However, there are currently no marketed drugs targeting IL-10. IL-10 can promote the expansion and survival of CD8+ T cells specific to specific antigens, and there is a positive correlation between specific antigen CD8+ T cells and immune cells in tumor killing. Although multiple studies have shown that immunomodulators can be used to exert anti-tumor effects in animal models and cancer patients, the short half-life and systemic toxicity associated with the application of immunomodulators greatly limit their use. After binding to its receptor, IL-10 activates the STAT3 and STATI pathways, which are the signal transduction pathways through which IL-10 exerts its biological function.

A fusion protein containing unmutated IL-10 is provided in patent CN201380041222.1. However, this fusion protein has low expression, low purity, weak binding activity and signal activation ability at the IL-10 end, and poor binding activity with cancer cells, and cannot be used for large-scale industrial production. Therefore, it is necessary to find proteins with both good binding activity to IL-10 and tumor-associated antigens.

### SUMMARY

The purpose of the present invention is to provide a heterodimeric fusion protein and use thereof. The heterodimeric fusion protein has a high affinity for both tumor antigens or immune checkpoints and IL-10 receptors, and has good anti-tumor activity.

The present invention provides a heterodimeric fusion protein, wherein the heterodimeric fusion protein comprises: a first heavy chain, wherein the first heavy chain comprises an Fc region, an immunomodulator fused to the Fc region; a light chain and a second heavy chain, wherein the light chain and the second heavy chain are complexed to form a targeting portion exhibiting binding specificity to a tumor antigen or an immune checkpoint; and wherein the light chain, the first heavy chain, and the second heavy chain are complexed to form the heterodimeric fusion protein, wherein the immunomodulator in the first heavy chain may contain a mutation.

In one embodiment, the immunomodulator is a cytokine, a cytokine receptor, a growth factor, a hormone, or an extracellular matrix molecule. In one embodiment, the immunomodulator is selected from the group consisting of: IL-1, IL-2, IL-2 Rα, IL-2 Rβ, IL-3, IL-3 Rα, IL-4, IL-4 Rα, IL-5, IL-5 Rα, IL-6, IL-6 Rα, IL-7, IL-7 Rα, IL-8, IL-9, IL-9 Rα, IL-10, IL-10R1, IL-10R2, IL-11, IL-11 Rα, IL-12, IL-12 Rα, IL-12 Rβ2, IL-12 Rβ1, IL-13, IL-13 Rα, IL-13 Rα2, IL-14, IL-15, IL-15Ra sushi, IL-16, IL-17, IL-18, IL-19, IL-20, IL-20R1, IL-20R2, IL-21, IL-21 Rα, IL-22, IL-23, IL-23R, IL-27 R, and IL-31 R. In one embodiment, the immunomodulator is IL-10.

In one embodiment, the IL-10 has an amino acid sequence as shown in SEQ ID NO: 1 or 2, or is an amino acid sequence having at least 80% identity to SEQ ID NO:1 or 2.

In one embodiment, the IL-10 has an amino acid sequence as shown in SEQ ID NO: 1, which may comprise one or more of the following mutation sites: N18Y, R104W, N92Q, T100D.

In one embodiment, the tumor antigen or immune checkpoint is one or more of B7H3, B7H4, B7H5, BTLA, CD27, CD28, CD153, CD40, CD40L, CD70, CD80, CD86, CD96, CD112, CD134, CD137, CD137L, CD152/CTLA-4, CD155, CD223, CD226, CD252/OX40L, CD258, CD273/PD-L2, CD274/PD-L1, CD278, CD279, CD357, DR3, Galectin-9, GITRL, HVEM, ICOSL/B7RP1/B7H2, IDO, TIGIT, TIM-3, TL1A, MART-1/MelanA, gp100, tyrosinase, TRP-1, TRP-2, MAGE-1, MAGE-3, BAGE, GAGE-1, GAGE-2, p15, CEA, p53, Ras, HER-2/neu, BCR-ABL, E2A-PRL, H4-RET, IGH-IGK, MYL-RAR, Epstein Barr Virus Antigen EBVA, Human papillomavirus antigen E6 or E7, TSP-180, MAGE-4, MAGE-5, MAGE-6, RAGE, NY-ESO, erbB, p185erbB2, p180erbB-3, c-met, nm-23H1, PSA, TAG-72, CA 19-9, CA 72-4, CAM 17.1, NuMa, K-ras, B-Cyclin, CDK4, Mum-1, p 15, p 16, 43-9F, 5T4, 791Tgp72, alpha fetoprotein, B-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 Binding Protein\Cyclophilin C-Associated Protein, TAAL6, TAG72, TLP, MUC16, IL13Rα2, FRα, VEGFR2, Lewis Y, FAP, EphA2, CEACAM5, CEACAM6, EGFR, CA6, CA9, GPNMB, EGP1, FOLR1, endothelial receptor, STEAP1, SLC44A4, Bindin-4, AGS-16, Guanylyl cyclase C, MUC-1, CFC1B, alpha 3 chain of integrin, TPS, CD19, CD20, CD22, CD30, CD72, CD180, CD171, CD123, CD133, CD138, CD37, CD70, CD79a, CD79b, CD56, CD74, CD166, CD71, CLL-1/CLEC12A, ROR1, Phosphatidylinositol proteoglycan 3, mesothelin, CD33/IL3Rα, c-Met, PSCA, PSMA, Glycolipid F77, EGFRVIII, BCMA, GD-2, MY-ESO-1 or MAGE A3.

In one embodiment, the light chain comprises a complementary decision region (CDR), the complementary decision region comprising an amino acid sequence having at least 80% identity to the amino acid sequence of the corresponding CDR of the light chain of the antibody that specifically binds a tumor antigen or an immune checkpoint.

In one embodiment, the light chain of the antibody that specifically binds to a tumor antigen or an immune checkpoint comprises the amino acid sequence of LCDR1 as shown in SEQ ID NO: 6, LCDR2 as shown in SEQ ID NO: 7, and LCDR3 as shown in SEQ ID NO: 8.

In one embodiment, the light chain comprises a variable region, the variable region comprising an amino acid sequence having at least 80% identity to an amino acid sequence contained in the variable region of the light chain of an antibody specifically directed against a tumor antigen or an immune checkpoint.

In one embodiment, the amino acid sequence of the variable region of the light chain is as shown in SEQ ID NO: 10, or is an amino acid sequence having at least 80% identity to SEQ ID NO: 10.

In one embodiment, the light chain has an amino acid sequence as shown in SEQ ID NO: 13, or is an amino acid sequence having at least 80% identity to SEQ ID NO: 13.

In one embodiment, the second heavy chain comprises a complementary determining region (CDR), the complementary determining region comprising an amino acid sequence having at least 80% identity to the amino acid sequence of the corresponding CDR of the second heavy chain of the antibody that specifically binds to a tumor antigen or an immune checkpoint.

In one embodiment, the second heavy chain of the antibody that specifically binds to a tumor antigen or an immune checkpoint comprises the amino acid sequence of HCDR1 as shown in SEQ ID NO: 3, HCDR2 as shown in SEQ ID NO: 4, and HCDR3 as shown in SEQ ID NO: 5.

In one embodiment, the second heavy chain comprises a variable region, the variable region comprising an amino acid sequence having at least 80% identity to an amino acid sequence contained in a light chain variable region of an antibody specifically directed against a tumor antigen or an immune checkpoint.

In one embodiment, the amino acid sequence of the variable region of the second heavy chain is as shown in SEQ ID NO: 9, or is an amino acid sequence having at least 80% identity to SEQ ID NO: 9.

In one embodiment, the amino acid sequence of the second heavy chain is as shown in SEQ ID NO: 12, or is an amino acid sequence having at least 80% identity to SEQ ID NO: 12.

In one embodiment, the amino acid sequence of the first heavy chain is as shown in SEQ ID NO: 15, or is an amino acid sequence having at least 80% identity to SEQ ID NO: 15.

In one embodiment, the immunomodulator is linked to the Fc region of the antibody that specifically binds to a tumor antigen or an immune checkpoint.

In one embodiment, the first heavy chain comprises a constant region of an immunoglobulin selected from IgG1, IgG2, IgG3 and IgG4.

In one embodiment, the first heavy chain comprises one or more Fc regions of the same or different types, and the Fc region are fused to the immunomodulator via a polypeptide linker. In one embodiment, the immunomodulator is linked to the N-terminus of the Fc region via a polypeptide linker. In one embodiment, the polypeptide linker is 5-30 amino acids. In one embodiment, the polypeptide linker is (GGGGS)n, wherein n=1-6.

In one embodiment, the first heavy chain comprises one or more immunomodulators of the same or different types, and the one or more immunomodulators are fused to each other and to the Fc region.

The present invention also provides a method of preparing the heterodimeric fusion protein, which is to transfer three recombinant plasmids containing the light chain, the first heavy chain and the second heavy chain respectively into the same host cell for recombinant expression. In one embodiment, the host cell is a mammalian cell, bacterial, fungal, or insect cell. In one embodiment, the mammalian cell is a CHO cell, SP20 cell, NSO cell, COS cell, BHK cell, HEK293 cell, or PerC6 cell. In one embodiment, the mammalian cell is a CHO cell.

The present invention also provides a nucleic acid encoding the heterodimeric fusion protein as described above. The present invention also provides a vector or plasmid containing the nucleic acid as described above. The present invention also provides a cell expressing the vector or plasmid as described above.

The present invention also provides a pharmaceutical composition comprising the heterodimeric fusion protein as described above and at least one pharmaceutically acceptable excipient, diluent or carrier. In one embodiment, the pharmaceutical composition can be used alone or in combination with other therapeutic agents to enhance efficacy or reduce potential side effects.

The present invention also provides the use of the heterodimeric fusion proteins in the preparation of drugs for the treatment of oncological diseases. In one embodiment, the tumor disease comprises colorectal cancer, membrane adenocarcinoma, lung cancer, esophageal cancer, prostate cancer, pro-connective tissue proliferative small round cell tumor, ovarian cancer, gastric cancer, pancreatic cancer, liver cancer, kidney cancer, breast cancer, non-small cell lung cancer, melanoma, alveolar rhabdomyosarcoma, embryonal rhabdomyosarcoma, Ewing sarcoma, nephroblastoma, neuroblastoma, ganglioneuroblastoma, medulloblastoma, high-grade glioma, diffuse intrinsic pontine glioma, and one or more of multi-layered chrysoidal mass embryonal tumors.

The present invention also provides the use of the above heterodimeric fusion proteins in the preparation of reagents or kits for the detection of tumor antigens or immune checkpoints and IL-10 receptor molecules.

The invention is further described below in connection with embodiments which are used to describe some specific embodiments of the invention and are not intended to limit the scope of protection of the invention. Unless otherwise defined, all technical and scientific terms used herein have the same meaning as those generally understood by ordinary technicians in the field to which this application belongs. Although methods and materials similar to or equivalent to the methods and materials described herein may be used in the practice or testing of this application, appropriate methods and materials are described below. In case of contradiction, the patent specification shall prevail.

The term “heterodimer”, generally refers to a molecule (e.g. a protein molecule) that consists of two different members. The two members of a heterodimer may differ in structure, function, activity and/or composition. For example, the two different members may contain polypeptides that differ in the order, number or type of amino acid residues that form these polypeptides. Each of the two different members of the heterodimer may independently comprise one, two or more units, polypeptide chains or moieties.

The term “targeting moiety”, generally refers to a molecule, complex or aggregate that specifically, selectively or preferentially binds to a target molecule, cell, particle, tissue or aggregate. For example, the targeting moiety may be an antibody, an antigen-binding antibody fragment, a bispecific antibody or any other antibody-based molecule or compound. Other examples of targeting moieties include, but are not limited to, aptamers, high affinity multimers, receptor binding ligands, nucleic acids, biotin affinity binding pairs, binding peptides or proteins, etc.

The terms “antigen binding site” and “binding portion”, generally refers to the portion of an antibody that is involved in antigen binding. The antigen binding site can be formed by amino acid residues in the N-terminal variable (“V”) region of the heavy (“H”) and/or light (“L”) chains. The three highly variable regions within the V-region of the heavy and light chains are called “hypervariable region” and are inserted between more conserved flanking regions called “framework regions” or “FRs”. In the antibody molecule, the three hypervariable regions of the light chain and the heavy chain are arranged opposite each other in three dimensions to form an antigen-binding ‘surface’. This surface mediates the recognition and binding of the target antigen.

Kabat is the most commonly used and defines CDRs based on sequence variability; Chothia defines CDRs based on sequence variability based on the position of the structural loop region; the IMGT system defines CDRs based on sequence variability and position within the variable domain structure; AbM is based on Oxford Molecular's AbM antibody modelling software and is a compromise between Kabat and Chothia; Contact defines the CDR based on the analysis of complex crystal structures and is similar to Chothia in several respects. Numbering of amino acid positions (e.g. amino acid residues in the Fc region) and target regions (e.g. CDR) in the present invention, using the Kabat system.

The term “tumor antigen”, generally refers to an antigenic substance produced in or by tumor cells that may have the ability to trigger an immune response in the host. For example, a tumor antigen may be a protein, polypeptide, peptide or fragment thereof that forms part of a tumor cell and is capable of inducing tumor-specific cytotoxic T lymphocytes. In some embodiments, the term “tumor antigen” may also refer to a biomolecule (e.g., protein, carbohydrate, glycoprotein, etc.) that is uniquely or preferentially or differentially expressed on and/or found to be associated with cancer cells and thus provides a preferential or specific target for cancer. For example, preferential expression may be preferential expression relative to any other cell in the organism, or preferential expression within a specific region of the organism (e.g. within a specific organ or tissue).

The term “immune checkpoint”, generally refers to suppressor and activator molecules in the immune system that regulate the body's anti-tumor immune system through modulation of T-cell activity. For example, suppressor molecules comprise PDL1, B7H3, CTLA4, etc. and activator molecules comprise OX40, 4-1BB, CD40, etc.

The term “Immunomodulator”, generally refers to substances that affect the function of the immune system. Immunomodulators may enhance or reduce the immune response. For example, immunomodulators can be active agents in immunotherapy, including, but not limited to, recombinant, synthetic and/or natural preparations of, for example, cytokines, granulocyte colony-stimulating factor (G-CSF), interferon, imiquimod, bacterial cell membrane fragments, chemokines, interleukins, cytosine phosphate-guanosine (CpG) oligodeoxynucleotides and dextran. In some embodiments, the immunomodulator is a cytokine.

The term “polypeptide linker” as used herein, generally refers to a synthetic amino acid sequence that links or couples two peptide sequences (e.g. links two peptide structural domains). A polypeptide linker may link two amino acid sequences through a peptide bond. In some embodiments, the polypeptide linker of the present application links an immunomodulator to the Fc region.

The term “antibody” as used herein, generally refers to a protein comprising one or more polypeptides substantially encoded by an immunoglobulin gene or fragment of an immunoglobulin gene. Immunoglobulin genes may include, κ, λ, α, γ, δ, ε and μ constant region genes and a variety of immunoglobulin variable region genes. As used herein, the light chain may be classified as κ or λ. The heavy chain may be classified as γ, μ, α, δ or ε, which in turn define the immunoglobulin classes: IgG, IgM, IgA, IgD and IgE, respectively. Antibodies as used herein may have structural units comprising tetramers. Each tetramer may comprise two pairs of identical polypeptide chains, each pair having a “light” chain (approximately 25 kD) and a “heavy” chain (approximately 50-70 kD). The N-terminus of each member may define a variable region of approximately 100 to 110 or more amino acids which is primarily responsible for antigen recognition. As used herein, the terms variable light chain (VL) and variable heavy chain (VH) usually refer to these regions of the light and heavy chains, respectively. Antibodies may exist as intact immunoglobulins or as a variety of fully characterised fragments produced by digestion with various polypeptidases or by de novo expression. The term “antibody” as used herein, may also include antibody fragments produced by modification of the whole antibody or by de novo synthesis using recombinant DNA methods, including but not limited to Fab′2, IgG, IgM, IgA, IgE, scFv, dAb, nanobodies, single and double chain antibodies. In some embodiments, the antibodies comprise, but are not limited to, Fab′2, IgG, IgM, IgA, IgE and single chain antibodies, such as single chain Fv (scFv) antibodies, wherein the variable heavy chain and variable light chain are linked together (either directly or via peptide junctions) to form a continuous polypeptide. In some embodiments, the antibodies and fragments of the present application are bispecific. In some embodiments, the bispecific antibody or fragment thereof has binding specificity for at least two different epitopes (e.g., at least one of the at least two different epitopes is a tumor-associated antigen). In some embodiments, the antibodies and fragments may also be heterologous antibodies, e.g. they may be or may comprise two or more linked antibodies or antibody-binding fragments (e.g. Fab), wherein each antibody or fragment has a different specificity.

The term ‘identity’ is defined as the percentage of amino acid residues in a candidate sequence that are identical to amino acid residues in a control polypeptide sequence after comparing the sequences and introducing gaps where necessary to obtain the maximum percentage sequence identity. Comparisons for the purpose of determining percentage amino acid sequence identity can be performed in a variety of ways within the skill of the art, for example, using publicly available computer software, such as BLAST software or the FASTA programme package.

The term ‘at least 80% identity’ means that the percentage of amino acid residues in the candidate sequence that are identical to amino acid residues in the control polypeptide sequence is 80% or more, including 80%, 81%, 82%, 83%, 84%, 85%, 86%, 87%, 88%, 89%, 90%, 91%, 92%, 93%, 94%, 95%, 96%, 97%, 98%, 99%, 100%.

The term “host cell”, generally comprises a single cell, cell line or cell culture that will be or has been a recipient of a subject's plasmid or vector comprising a polynucleotide as disclosed herein or expressing a proteinaceous heterodimer of the present application. The host cell may consist of the progeny of a single host cell. The progeny may not necessarily be identical (morphologically or in terms of total genomic DNA complement) to the original parental cells due to natural, accidental or intentional mutations. The host cell may be cells transfected in vitro with a vector as disclosed in this application. The host cells may be bacterial cells (e.g. Escherichia coli (E. coli)), yeast cells or other eukaryotic cells such as COS cells, Chinese hamster ovary (CHO) cells, HeLa cells or myeloma cells.

The term “vector” as used herein, generally refers to a nucleic acid molecule capable of self-replication in a suitable host, which transfers the inserted nucleic acid molecule into and/or between host cells. The term may include vectors primarily for insertion of DNA or RNA into a cell, vectors primarily for replication of DNA or RNA, and expression vectors for transcription and/or translation of DNA or RNA. It also comprises vectors that perform more than one of these functions. An “expression vector” is a polynucleotide that can be transcribed and translated into a polypeptide when introduced into a suitable host cell.

The terms “treatment” refer to methods of achieving beneficial or desired results including, but not limited to, therapeutic benefit and/or preventive benefit. Therapeutic benefit generally refers to the elimination or reduction in severity of the underlying condition being treated. In addition, therapeutic benefit is achieved by eliminating, reducing the severity of, or reducing the incidence of one or more physical symptoms associated with the underlying condition such that an improvement is observed in the subject (although the subject may still be afflicted with the underlying condition). For preventive benefits, the combination may be administered to subjects at risk of developing a specific disease, or who report one or more physical symptoms of the disease, even though a diagnosis of the disease may not have been made.

The term “in vivo”, generally refers to events that occur within the body of the subject.

The term “in vitro” as used herein, generally refers to events that occur outside the body of the subject. For example, in vitro assays include any assay that is performed outside the subject. In vitro assays include cell-based assays that use dead or living cells. In vitro assays also include cell-free assays that do not use intact cells.

The term “subject”, generally refers to human or non-human animals, including but not limited to cats, dogs, horses, pigs, cattle, sheep, goats, rabbits, mice, rats or monkeys.

The amino acid sequences of the heterodimeric fusion proteins (hereinafter referred to as ‘fusion proteins’), IL-10 and variants thereof, and the control antibodies referred to in the Examples are shown in Table 1. The structures of fusion proteins 1 and 2 are shown in FIG. 1, differing only in that IL-10 is natural in fusion protein 1 and mutated in fusion protein 2.

TABLE 1
Amino acid sequences of the fusion proteins and IL10, etc. of
the present invention.

| Name | Amino acid or nucleotide sequence |
|---|---|
| IL-10 | Signalling peptide - natural IL-10: |
| | MHSSALLCCLVLLTGVRASPGQGTQSENSCTHFPGNLPNMLR |
| | DLRDAFSRVKTFFQMKDQLDNLLLKESLLEDFKGYLGCQALS |
| | EMIQFYLEEVMPQAENQDPDIKAHVNSLGENLKTLRLRLRRC |
| | HRFLPCENKSKAVEQVKNAFNKLQEKGIYKAMSEFDIFINYIE |
| | AYMTMKIRN (SEQ ID NO: 1) |
| | Signalling peptide - mutated IL-10: |
| | MHSSALLCCLVLLTGVRASPGQGTQSENSCTHFPGYLPNMLR |
| | DLRDAFSRVKTFFQMKDQLDNLLLKESLLEDFKGYLGCQALS |
| | EMIQFYLEEVMPQAENQDPDIKAHVQSLGENLKDLRLWLRRC |
| | HRFLPCENKSKAVEQVKNAFNKLQEKGIYKAMSEFDIFINYIE |
| | AYMTMKIRN (SEQ ID NO: 2) |
| Fusion protein | the second heavy chain VH CDR1: DTYIH (SEQ ID NO: 3) |
| 1 (HER2 + IL10 | the second heavy chain VH CDR2: RIYPT (SEQ ID NO: 4) |
| wild) | the second heavy chain VH CDR3: WGGDGFYAMDY (SEQ ID |
| | NO: 5) |
| | the light chain VL CDR1: RASQDVNTAVA (SEQ ID NO: 6) |
| | the light chain VL CDR2: SASFLYS (SEQ ID NO: 7) |
| | the light chain VL CDR3: QQHYTTPPT (SEQ ID NO: 8) |
| | Variable region of the second heavy chain: |
| | EVQLVESGGGLVQPGGSLRLSCAASGFNIKDTYIHWVRQAPG |
| | KGLEWVARIYPTNGYTRYADSVKGRFTISADTSKNTAYLQMN |
| | SLRAEDTAVYYCSRWGGDGFYAMDYWGQGTLVTVSS(SEQ |
| | ID NO: 9) |
| | Variable region of the light chain: |
| | DIQMTQSPSSLSASVGDRVTITCRASQDVNTAVAWYQQKPGK |
| | APKLLIYSASFLYSGVPSRFSGSRSGTDFTLTISSLQPEDFATYY |
| | CQQHYTTPPTFGQGTKVEIK (SEQ ID NO: 10) |
| | Fc region of the first heavy chain: |
| | DKTHTCPPCPAPELLGGPSVFLFPPKPKDTLMISRTPEVTCVVV |
| | DVSHEDPEVKFNWYVDGVEVHNAKTKPREEQYNSTYRVVSV |
| | LTVLHQDWLNGKEYKCKVSNKALPAPIEKTISKAKGQPREPQ |
| | VYTLPPSRDELTKNQVSLTCLVKGFYPSDIAVEWESNGQPENN |
| | YKTTPPVLDSDGSFFLASKLTVDKSRWQQGNVFSCSVMHEAL |
| | HNHYTQKSLSLSPGK (SEQ ID NO: 11) |
| | Full length of the second heavy chain: |
| | EVQLVESGGGLVQPGGSLRLSCAASGFNIKDTYIHWVRQAPG |
| | KGLEWVARIYPTNGYTRYADSVKGRFTISADTSKNTAYLQMN |
| | SLRAEDTAVYYCSRWGGDGFYAMDYWGQGTLVTVSSASTK |
| | GPSVFPLAPSSKSTSGGTAALGCLVKDYFPEPVTVSWNSGALT |
| | SGVHTFPAVLQSSGLYSLSSVVTVPSSSLGTQTYICNVNHKPSN |
| | TKVDKKVEPKSCDKTHTCPPCPAPELLGGPSVFLFPPKPKDTL |
| | MISRTPEVTCVVVDVSHEDPEVKFNWYVDGVEVHNAKTKPR |
| | EEQYNSTYRVVSVLTVLHQDWLNGKEYKCKVSNKALPAPIEK |
| | TISKAKGQPREPQVYTLPPSRDELTKNQVSLWCLVKGFYPSDI |
| | AVEWESNGQPENNYKTTPPVLDSDGSFFLYSKLTVDKSRWQQ |
| | GNVFSCSVMHEALHNHYTQKSLSLSPGK |
| | (SEQ ID NO: 12) |
| | Full length of the light chain: |
| | DIQMTQSPSSLSASVGDRVTITCRASQDVNTAVAWYQQKPGK |
| | APKLLIYSASFLYSGVPSRFSGSRSGTDFTLTISSLOPEDFATYY |
| | CQQHYTTPPTFGQGTKVEIKRTVAAPSVFIFPPSDEQLKSGTAS |
| | VVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 13) |
| | Full length of the first heavy chain: |
| | SPGQGTQSENSCTHFPGNLPNMLRDLRDAFSRVKTFFQMKDQ |
| | LDNLLLKESLLEDFKGYLGCQALSEMIQFYLEEVMPQAENQD |
| | PDIKAHVNSLGENLKTLRLRLRRCHRFLPCENKSKAVEQVKN |
| | AFNKLQEKGIYKAMSEFDIFINYIEAYMTMKIRNGGGGSGGGG |
| | SGGGGSSPGQGTQSENSCTHFPGNLPNMLRDLRDAFSRVKTFF |
| | QMKDQLDNLLLKESLLEDFKGYLGCQALSEMIQFYLEEVMPQ |
| | AENQDPDIKAHVNSLGENLKTLRLRLRRCHRFLPCENKSKAV |
| | EQVKNAFNKLQEKGIYKAMSEFDIFINYIEAYMTMKIRNGGG |
| | GSDKTHTCPPCPAPELLGGPSVFLFPPKPKDTLMISRTPEVTCV |
| | VVDVSHEDPEVKFNWYVDGVEVHNAKTKPREEQYNSTYRVV |
| | SVLTVLHQDWLNGKEYKCKVSNKALPAPIEKTISKAKGQPRE |
| | PQVYTLPPSRDELTKNQVSLTCLVKGFYPSDIAVEWESNGQPE |
| | NNYKTTPPVLDSDGSFFLASKLTVDKSRWQQGNVFSCSVMHE |
| | ALHNHYTQKSLSLSPGK (SEQ ID NO: 14) |
| Fusion protein | the second heavy chain VH CDR1: DTYIH (SEQ ID NO: 3) |
| 2 (HER2 + IL10 | the second heavy chain VH CDR2: RIYPT (SEQ ID NO: 4) |
| mutated) | the second heavy chain VH CDR3: WGGDGFYAMDY (SEQ ID |
| | NO: 5) |
| | the light chain VL CDR1: RASQDVNTAVA (SEQ ID NO: 6) |
| | the light chain VL CDR2: SASFLYS (SEQ ID NO: 7) |
| | the light chain VL CDR3: QQHYTTPPT (SEQ ID NO: 8) |
| | Variable region of the second heavy chain: |
| | EVQLVESGGGLVQPGGSLRLSCAASGFNIKDTYIHWVRQAPG |
| | KGLEWVARIYPTNGYTRYADSVKGRFTISADTSKNTAYLQMN |
| | SLRAEDTAVYYCSRWGGDGFYAMDYWGQGTLVTVSS (SEQ |
| | ID NO: 9) |
| | Variable region of the light chain: |
| | DIQMTQSPSSLSASVGDRVTITCRASQDVNTAVAWYQQKPGK |
| | APKLLIYSASFLYSGVPSRFSGSRSGTDFTLTISSLOPEDFATYY |
| | CQQHYTTPPTFGQGTKVEIK (SEQ ID NO: 10) |
| | Fc region of the first heavy chain: |
| | DKTHTCPPCPAPELLGGPSVFLFPPKPKDTLMISRTPEVTCVVV |
| | DVSHEDPEVKFNWYVDGVEVHNAKTKPREEQYNSTYRVVSV |
| | LTVLHQDWLNGKEYKCKVSNKALPAPIEKTISKAKGQPREPQ |
| | VYTLPPSRDELTKNQVSLTCLVKGFYPSDIAVEWESNGQPENN |
| | YKTTPPVLDSDGSFFLASKLTVDKSRWQQGNVFSCSVMHEAL |
| | HNHYTQKSLSLSPGK (SEQ ID NO: 11) |
| | Full length of the second heavy chain: |
| | EVQLVESGGGLVQPGGSLRLSCAASGFNIKDTYIHWVRQAPG |
| | KGLEWVARIYPTNGYTRYADSVKGRFTISADTSKNTAYLQMN |
| | SLRAEDTAVYYCSRWGGDGFYAMDYWGQGTLVTVSSASTK |
| | GPSVFPLAPSSKSTSGGTAALGCLVKDYFPEPVTVSWNSGALT |
| | SGVHTFPAVLQSSGLYSLSSVVTVPSSSLGTQTYICNVNHKPSN |
| | TKVDKKVEPKSCDKTHTCPPCPAPELLGGPSVFLFPPKPKDTL |
| | MISRTPEVTCVVVDVSHEDPEVKFNWYVDGVEVHNAKTKPR |
| | EEQYNSTYRVVSVLTVLHQDWLNGKEYKCKVSNKALPAPIEK |
| | TISKAKGQPREPQVYTLPPSRDELTKNQVSLWCLVKGFYPSDI |
| | AVEWESNGQPENNYKTTPPVLDSDGSFFLYSKLTVDKSRWQQ |
| | GNVFSCSVMHEALHNHYTQKSLSLSPGK (SEQ ID NO:12) |
| | Full length of the light chain: |
| | DIQMTQSPSSLSASVGDRVTITCRASQDVNTAVAWYQQKPGK |
| | APKLLIYSASFLYSGVPSRFSGSRSGTDFTLTISSLQPEDFATYY |
| | CQQHYTTPPTFGQGTKVEIKRTVAAPSVFIFPPSDEQLKSGTAS |
| | VVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 13) |
| | Full length of the first heavy chain: |
| | SPGQGTQSENSCTHFPGYLPNMLRDLRDAFSRVKTFFQMKDQ |
| | LDNLLLKESLLEDFKGYLGCQALSEMIQFYLEEVMPQAENQD |
| | PDIKAHVQSLGENLKDLRLWLRRCHRFLPCENKSKAVEQVKN |
| | AFNKLQEKGIYKAMSEFDIFINYIEAYMTMKIRNGGGGSGGGG |
| | SGGGGSSPGQGTQSENSCTHFPGYLPNMLRDLRDAFSRVKTFF |
| | QMKDQLDNLLLKESLLEDFKGYLGCQALSEMIQFYLEEVMPQ |
| | AENQDPDIKAHVQSLGENLKDLRLWLRRCHRFLPCENKSKAV |
| | EQVKNAFNKLQEKGIYKAMSEFDIFINYIEAYMTMKIRNGGG |
| | GSDKTHTCPPCPAPELLGGPSVFLFPPKPKDTLMISRTPEVTCV |
| | VVDVSHEDPEVKFNWYVDGVEVHNAKTKPREEQYNSTYRVV |
| | SVLTVLHQDWLNGKEYKCKVSNKALPAPIEKTISKAKGQPRE |
| | PQVYTLPPSRDELTKNQVSLTCLVKGFYPSDIAVEWESNGQPE |
| | NNYKTTPPVLDSDGSFFLASKLTVDKSRWQQGNVFSCSVMHE |
| | ALHNHYTQKSLSLSPGK (SEQ ID NO: 15) |
| Negative | Amino acid sequence of the heavy chain: |
| control | QVQLVQSGAEVKKPGASVKVSCKASGYTFTDYAMHWVRQA |
| (NEO-201 | PGQRLEWMGLISTYSGDTKYNQNFQGRVTMTVDKSASTAYM |
| antibody | ELSSLRSEDTAVYYCARGDYSGSRYWFAYWGQGTLVTVSSAS |
| msIgG1 | TKGPSVFPLAPSSKSTSGGTAALGCLVKDYFPEPVTVSWNSGA |
| subtype) | LTSGVHTFPAVLQSSGLYSLSSVVTVPSSSLGTQTYICNVNHKP |
| | SNTKVDKKVEPKSCDKTHTCPPCPAPELLGGPSVFLFPPKPKD |
| | TLMISRTPEVTCVVVDVSHEDPEVKFNWYVDGVEVHNAKTK |
| | PREEQYNSTYRVVSVLTVLHQDWLNGKEYKCKVSNKALPAPI |
| | EKTISKAKGQPREPQVYTLPPSRDELTKNQVSLTCLVKGFYPS |
| | DIAVEWESNGQPENNYKTTPPVLDSDGSFFLYSKLTVDKSRW |
| | QQGNVFSCSVMHEALHNHYTQKSLSLSPGK |
| | (SEQ ID NO: 16) |
| | Amino acid sequence of the light chain: |
| | DIQMTQSPSSLSASVGDRVTITCQASENIYGALNWYQRKPGKS |
| | PKLLIYGASNLATGMPSRFSGSGSGTDYTFTISSLQPEDIATYY |
| | CQQVLSSPYTFGGGTKLEIKRTVAAPSVFIFPPSDEQLKSGTAS |
| | VVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 17) |

### BRIEF DESCRIPTION OF THE DRAWINGS

FIG. 1: Schematic diagram of the heterodimeric fusion protein of the present invention.

FIG. 2: The binding activity of fusion proteins 1 and 2 to HER2 protein was detected by ELISA.

FIG. 3: The binding activity of fusion protein 2 to IL-10 receptor protein was detected by ELISA.

FIG. 4: The binding activity of the heterodimeric fusion proteins of the present invention to BT474 cells.

FIG. 5: IL-10 end luciferase expression activation validation of positive and negative controls.

FIG. 6: IL-10 end luciferase expression activation validation of fusion protein 1.

FIG. 7: IL-10 end luciferase expression activation validation of fusion protein 2.

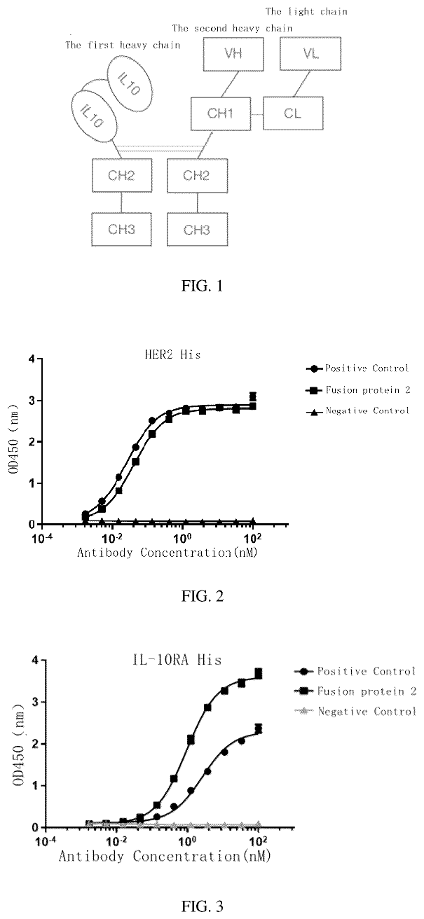

### DETAILED DESCRIPTION

### Example 1 Gene Synthesis and Construction of Expression Vector

The PcDNA3.1 vector was used as a dedicated vector for expressing the light and heavy chains of the fusion protein. The pcDNA3.1 vector comprises the CMV Promoter used by the heavy chain, the eukaryotic screening marker G418 tag and the prokaryotic screening tag Ampicilline. The nucleotide sequences (i.e. target genes) of the fusion protein-expressing the first heavy chain, the second heavy chain and the light chain coding genes were obtained separately by gene synthesis. The vector and the target fragment were double-enzyme-cut with HindIII and XhoI. After recovery, the DNA ligase was used for enzyme ligation, and the E. coli competent cell DH5a was transformed. The positive clones were selected and subjected to plasmid extraction and restriction digestion verification. The recombinant plasmids containing the genes encoding the first heavy chain, the second heavy chain and the light chain of the fusion protein was obtained.

### Example 2 Plasmid Extraction

According to the method described in “Molecular Cloning Experiment Guide” (2002, Science Press), the recombinant plasmids containing the above-mentioned target genes were transformed into competent E. coli cells DH5a, and the transformed bacteria were spread and cultured on LB plates containing 100 μg/mL ampicillin. The plasmid clones were selected and cultured in liquid LB medium, shaken at 260 rpm for 14 hours, and the plasmids were extracted from the endotoxin-free plasmid large extraction kit, dissolved in sterile water, and the concentration was determined with a nucleic acid protein quantifier.

### Example 3 Plasmid Transfection, Transient Expression and Fusion Protein Purification

ExpiCHO was cultured to a cell density of 6×10⁶ cells/mL at 37° C., 8% CO₂ and 100 rpm. The constructed vector plasmids were transfected into the above cells at a mass concentration of 1:1:1 by liposomes. The transfection plasmid concentration was 1 mg/mL, and the liposome concentration was determined by reference to the ExpiCHO™ Expression System kit. It was incubated at 32° C., 5% CO₂ and 100 rpm for 7-10 days. Feeding was given after 18-22 h and during 5-8 days after transfection. The above culture products were centrifuged at 4000 g in a centrifuge, filtered with a 0.22 μm filter membrane, and the culture supernatant was collected. The obtained antibody proteins were purified with Protein A and ion column, and the eluate was collected.

The specific operation steps of Protein A and ion column purification were as follows: the cell culture solution was centrifuged at a high speed and the supernatant was taken, and the protein A column of GE is used for affinity chromatography. The balance buffer for chromatography was 1×PBS (pH 7.4). After the cell supernatant was loaded and combined, the cell supernatant was washed with PBS until the ultraviolet rays returned to baseline. The target protein was eluted with 0.1 M glycine (pH 3.0) in the elution buffer, and then the pH was adjusted to neutral with Tris for storage. The pH of the product obtained by affinity chromatography was adjusted to be lower or higher than pI 1-2 pH units, and diluted appropriately to control the sample conductivity below 5 ms/cm. Through suitable pH buffer such as phosphate buffer, acetate buffer, etc., conventional ion exchange chromatography methods in the art such as anion exchange or cation exchange for NaCl gradient elution were used. According to SDS-PAGE, the collection tube where the target protein was located was selected, and the target protein was combined and stored. Then, the eluate obtained after purification was ultrafiltered and replaced into a buffer solution.

### Example 4 Detection of the Affinity of the Fusion Protein to HER2 by ELISA

huHER2-his (purchased from ACRO, CAT: HE2-H5225) was diluted to 0.5 μg/mL with PBS buffer at pH 7.4, and 100 μL per well was added to a 96-well ELISA plate and coated overnight at 4° C. It was blocked with 1% BSA blocking solution for 1 hour. After washing the plate 3 times with PBST, the purified fusion proteins were diluted to 100 nM with 0.5% BSA sample diluent as the starting concentration and subjected to a 3-fold gradient dilution for a total of 11 gradients, and a negative control (NEO-201 antibody, msIgG1 subtype, see CN 111670199 A, the heavy chain amino acid sequence is SEQ ID NO: 16, and the light chain amino acid sequence is SEQ ID NO: 17) and a positive control antibody (INN trastuzumab, or trastuzumab) were set up, and was incubated at 100 μL per well at 37° C. for 1 h. The plate was washed three more times with PBST and HRP-labelled goat anti-human IgG Fc (Jackson Cat: 109-035-098) was diluted with sample diluent at 1:10000, and 100 μL was added to each well, and incubated at room temperature for 1 h. After washing the plate 4 times with PBST, 100 μL of TMB substrate was added into each well and incubated at room temperature without light for 10 min. Then 100 μL of 1M HCl solution was added into each well to terminate the color development reaction. The absorbance value of each well in the 96-well plate was measured on a multifunctional enzyme standard at a wavelength of 450 nm and a reference wavelength of 570 nm. The absorbance value (OD) per well=OD450 nm-OD570 nm. The concentrations of the fusion proteins were logarithmically used as the horizontal coordinate, the measured absorbance values per well were used as the vertical coordinate. Sigmoidal dose-response (Variable Slope) method (Graph Pad Prism Software, Graph Pad Software, SanDiego, California) was chosen for non-linear regression to obtain the binding curve of fusion protein and HER2 protein.

The ELISA results for the fusion protein are shown in FIG. 2. The fusion protein 2 can bind to HER2 in multiple concentration ranges.

### Example 5 Detection of the Affinity of the Fusion Protein to the IL-10 Receptor by ELISA

The IL-10 receptor human-IL10RA-his (purchased from Sino Biological Inc., Beijing CAT: 10419-H08H) was diluted to 0.8 μg/mL with PBS buffer at pH 7.4, and 100 μL per well was added to a 96-well ELISA plate and coated overnight at 4° C. It was blocked with 1% BSA blocking solution for 1 hour. After washing the plate 3 times with PBST, the purified antibodies were diluted to 100 nM with 0.5% BSA sample diluent as the starting concentration and subjected to a 3-fold gradient dilution for a total of 11 gradients, and a negative control (INN trastuzumab, or trastuzumab) and a positive control (the fusion protein obtained in Example 4 of US202110141918.8) were set up, and was incubated at 100 μL per well at 37° C. for 1 h. The plate was washed three more times with PBST and HRP-labelled goat anti-human IgG Fc (Jackson Cat: 109-035-098) was diluted with sample diluent at 1:10000, and 100 μL was added to each well, and incubated at room temperature for 1 h. After washing the plate 4 times with PBST, 100 μL of TMB substrate was added into each well and incubated at room temperature without light for 10 min. Then 100 μL of 1M HCl solution was added into each well to terminate the color development reaction.

The absorbance value of each well in the 96-well plate was measured on a multifunctional enzyme standard at a wavelength of 450 nm and a reference wavelength of 570 nm. The absorbance value (OD) per well=OD450 nm-OD570 nm. The concentration of the antibody was logarithmically used as the horizontal coordinate, the measured absorbance value per well was used as the vertical coordinate, and the Sigmoidal dose-response (Variable Slope) method (Graph Pad Prism Software, Graph Pad Software, SanDiego, California) was selected for non-linear regression to obtain the binding curve between the target fusion protein and the IL-10 receptor IL-10RA protein.

The ELISA results for the fusion proteins are shown in FIG. 3. Fusion protein 2 can bind to the IL-10 receptor in multiple concentration ranges, which is superior to the positive control.

### Example 6 Binding Activity of the HER2 End of the IL-10 Bioactivity of the Fusion Protein to BT474 Cells

Morphologically normal BT474 cells (from Shanghai Chinese Academy of Sciences) with logarithmically growth were taken, and transferred to a centrifuge tube and centrifuged at 1000 rpm for 5 min. The cells were resuspended with FACS buffer at the density of 1×10⁶/mL, and 100 μL of the cells were dispensed into separate tubes. The purified fusion protein was diluted to 200 nM with FACS buffer as a starting concentration, and then diluted by 5-fold gradient dilution, with a total of 8 gradients, and negative control (i.e., NEO-201 antibody msIgG1 subtype, see CN111670199A, the heavy chain amino acid sequence is SEQ ID NO: 16, and the light chain amino acid sequence is SEQ ID NO: 17) and positive control (INN trastuzumab) were set up, and 100 μL of fusion protein dilution was added. The cells were incubated at 4° C. for 60 min and then washed twice with excess FACS buffer. The cells were resuspended in 100 μL of FACS buffer and goat anti-human secondary antibody-PE (Biolgend, Cat: 398004) was added to the sample, incubated for 30 min and washed twice with excess FACS buffer. The cells were immobilized in a fixed buffer and subsequently analysed by flow cytometry. The binding activity of the fusion protein to BT474 cells was detected by FACS method.

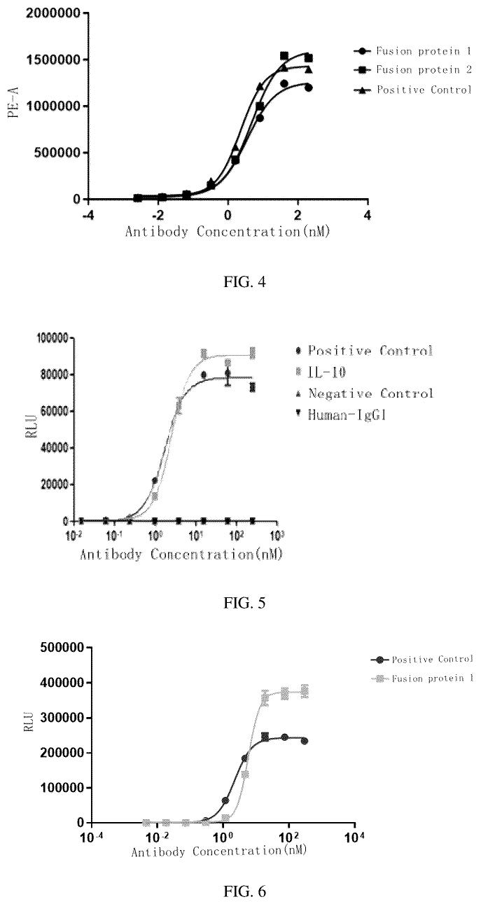

The results of the FACS assay for the binding activity of fusion proteins 1 and 2 to BT474 cells are shown in FIG. 4. Both fusion proteins 1 and 2 can bind specifically to BT474 in multiple concentration ranges.

### Example 7 Luciferase Expression Activation Validation of IL-10 End

Binding of IL-10 to IL-10R mediates the control of the degree and duration of inflammation, which is essential for maintaining homeostasis of the body's inflammatory response. This process is dependent on the regulation of signal transduction and activator of transcription 3 (STAT3). In this experiment, HEK293 tool cells were used to stably express IL-10R and the STAT3 signalling pathway reporter gene system, and IL-10 protein stimulation was used to activate the increase of cellular luciferase expression. Therefore, this cell line was used to study the activity of IL-10-STAT3.

IL-10-Reporter-HEK-293 cell line with logarithmic growth phase (purchased from Genomeditech Co. LTD.) was taken, and 2.5×10⁴ cells per well were spread in a 96-well cell culture plate (WHB, Cat: WHB-96-01), and cultured overnight at 37° C. in a 5% CO₂ incubator. Then positive control (fusion protein obtained in Example 4 of Patent 2021101419188), IL-10 protein (Novoprotein, Cat: CX04), negative control (Cetuximab), human-IgG1, fusion protein 1 and fusion protein 2 were diluted with dilution solution (DMEM complete medium) in 4-fold gradient from 300 μM respectively, with a total of 9 concentration gradients, and 100 μL/well were added into the supernatant-aspirated 96-well cell culture plate. After mixing, it was incubated at 37° C. and 5% CO₂ for 6 h. Then Bio-Lite Luciferase Assay substrate (Vazyme, Cat: DD1201-02) color developing solution was added at 100 μL/well and incubated at room temperature for 10 min. After that, the fluorescence value was detected by reading with an enzyme label.

Using the fusion protein concentration as the horizontal coordinate, the relative luminescence value (RLU) as the vertical coordinate, and the non-linear regression analysis method in GraphPad Prism software (Graphpad Prism 5 Demo, San Diego California), Log (agonist) v.s. Response—Variable Slope method was selected to fit the curve, and the fluorescence dose-effect curve of fusion protein was obtained.

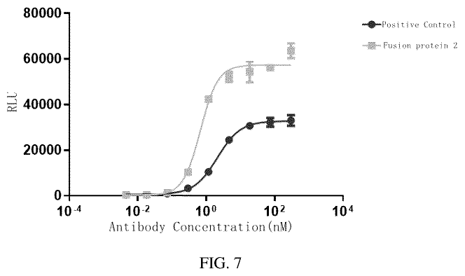

As shown in FIG. 5, IL-10 and the positive control activated luciferase expression in HEK293 cells in a dose-dependent manner, whereas the negative control cetuximab and human IgG1 had no such effect, indicating that this reporter gene method could reflect IL-10 signaling activation activity. The positive control EC50/fusion protein EC50 was calculated, and if the relative activity value was greater than 100%, it indicated that the activity of fusion protein was superior to that of the positive control, and conversely, the activity of fusion protein was lower than that of the positive control. In FIG. 6, the EC50 of the positive control and fusion protein 1 were 2.259 nM and 5.839 nM, respectively, and the relative activity value of the two was 39%. In FIG. 7, the EC50 of the positive control and fusion protein 2 were 2.117 nM and 0.648 nM, respectively, and the relative activity value of the two was 327%. It can be seen that the activation activity of fusion protein 2 is superior to that of fusion protein 1.

It should be noted that the above is only a preferred embodiment of the present invention and is not intended to limit the scope of the present invention. Any modifications, equivalent replacements and improvements etc. made within the spirit and principles of the present invention shall be included within the scope of protection of the present invention.

1. A heterodimeric fusion protein, comprising: a first heavy chain, wherein the first heavy chain comprises an Fc region, an immunomodulator fused to the Fc region; a light chain and a second heavy chain, wherein the light chain and the second heavy chain are complexed to form a targeting portion exhibiting binding specificity to a tumor antigen or an immune checkpoint; and wherein the light chain, the first heavy chain, and the second heavy chain are complexed to form the heterodimeric fusion protein.

2. The heterodimeric fusion protein of claim 1, wherein the immunomodulator is a cytokine, a cytokine receptor, a growth factor, a hormone, or an extracellular matrix molecule; preferably, the immunomodulator is selected from the group consisting of: IL-1, IL-2, IL-2 Rα, IL-2 Rβ, IL-3, IL-3 Rα, IL-4, IL-4 Rα, IL-5, IL-5 Rα, IL-6, IL-6 Rα, IL-7, IL-7 Rα, IL-8, IL-9, IL-9 Rα, IL-10, IL-10R1, IL-10R2, IL-11, IL-11 Rα, IL-12, IL-12 Rα, IL-12 Rβ2, IL-12 Rβ1, IL-13, IL-13 Rα, IL-13 Rα2, IL-14, IL-15, IL-15Ra sushi, IL-16, IL-17, IL-18, IL-19, IL-20, IL-20R1, IL-20R2, IL-21, IL-21 Rα, IL-22, IL-23, IL-23R, IL-27 R, and IL-31 R; more preferably, the immunomodulator is IL-10.

3. The heterodimeric fusion protein of claim 1, wherein the immunomodulator comprises at least 1-4 mutations.

4. The heterodimeric fusion protein of claim 3, wherein the site of the mutation comprises one or more of N18Y, R104W, N92Q, or T100D.

5. The heterodimeric fusion protein of claim 4, wherein the first heavy chain comprises one or more immunomodulators, and the immunomodulators are fused to each other and to the Fc region.

6. The heterodimeric fusion protein of claim 5, wherein the tumor antigen or immune checkpoint is one or more of B7H3, B7H4, B7H5, BTLA, CD27, CD28, CD153, CD40, CD40L, CD70, CD80, CD86, CD96, CD112, CD134, CD137, CD137L, CD152/CTLA-4, CD155, CD223, CD226, CD252/OX40L, CD258, CD273/PD-L2, CD274/PD-L1, CD278, CD279, CD357, DR3, Galectin-9, GITRL, HVEM, ICOSL/B7RP1/B7H2, IDO, TIGIT, TIM-3, TL1A, MART-1/MelanA, gp100, tyrosinase, TRP-1, TRP-2, MAGE-1, MAGE-3, BAGE, GAGE-1, GAGE-2, p15, CEA, p53, Ras, HER-2/neu, BCR-ABL, E2A-PRL, H4-RET, IGH-IGK, MYL-RAR, Epstein Barr Virus Antigen EBVA, Human papillomavirus antigen E6 or E7, TSP-180, MAGE-4, MAGE-5, MAGE-6, RAGE, NY-ESO, erbB, p185erbB2, p180erbB-3, c-met, nm-23H1, PSA, TAG-72, CA 19-9, CA 72-4, CAM 17.1, NuMa, K-ras, β-Cyclin, CDK4, Mum-1, p 15, p 16, 43-9F, 5T4, 791Tgp72, alpha fetoprotein, β-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 Binding Protein\Cyclophilin C-Associated Protein, TAAL6, TAG72, TLP, MUC16, IL13Rα2, FRα, VEGFR2, Lewis Y, FAP, EphA2, CEACAM5, CEACAM6, EGFR, CA6, CA9, GPNMB, EGP1, FOLR1, endothelial receptor, STEAP1, SLC44A4, Bindin-4, AGS-16, Guanylyl cyclase C, MUC-1, CFC1B, alpha 3 chain of integrin, TPS, CD19, CD20, CD22, CD30, CD72, CD180, CD171, CD123, CD133, CD138, CD37, CD70, CD79a, CD79b, CD56, CD74, CD166, CD71, CLL-1/CLEC12A, ROR1, Phosphatidylinositol proteoglycan 3, mesothelin, CD33/IL3Rα, c-Met, PSCA, PSMA, Glycolipid F77, EGFRvIII, BCMA, GD-2, MY-ESO-1 or MAGE A3.

7. The heterodimeric fusion protein of claim 6, wherein both the light chain and the second heavy chain comprise a complementary determining region, and the complementary determining region comprises an amino acid sequence having at least 80% identity to the amino acid sequence of the corresponding CDR of the light or heavy chain of the antibody that specifically binds the tumor antigen or the immune checkpoint.

8. (canceled)

9. A pharmaceutical composition, comprising the heterodimeric fusion protein of claim 1 and at least one pharmaceutically acceptable excipient, diluent or carrier.

10. Use of the heterodimeric fusion protein of claim 1 for: (a) preparing a drug for the treatment of an oncological disease, wherein the oncological disease comprises colorectal cancer, membrane adenocarcinoma, lung cancer, esophageal cancer, prostate cancer, pro-connective tissue proliferative small round cell tumor, ovarian cancer, gastric cancer, pancreatic cancer, liver cancer, kidney cancer, breast cancer, non-small cell lung cancer, melanoma, alveolar rhabdomyosarcoma, embryonal rhabdomyosarcoma, Ewing sarcoma, nephroblastoma, neuroblastoma, ganglioneuroblastoma, medulloblastoma, high-grade glioma, diffuse intrinsic pontine glioma, and one or more of multi-layered chrysoidal mass embryonal tumors; or (b) preparing reagents or kits for the detection of tumor antigens or immune checkpoints and IL-10 receptor molecules.

11. The heterodimeric fusion protein of claim 1, wherein the immunomodulator in the first heavy chain contains a mutation.
